(19) United States
(12) Patent Application Publication
(10) Pub. No.: US 2014/0249649 A1
(43) Pub. Date: Sep. 4, 2014

(54) PROSTHETIC LIMB ATTACHMENT SYSTEM

(71) Applicants: Daniel Berschinski, Washington, DC (US); Michael John Corcoran, Silver Spring, MD (US)

(72) Inventors: Daniel Berschinski, Washington, DC (US); Michael John Corcoran, Silver Spring, MD (US)

(21) Appl. No.: 13/784,527

(22) Filed: Mar. 4, 2013

(51) Int. Cl. A61F 2/80

(52) U.S. Cl. CPC A61F 2/80
USPC 623/35

(57) ABSTRACT

In one embodiment, a system for attaching a prosthetic limb to a user's lower torso includes a socket shaped to receive therein a leg amputation site of the user. The system also includes a flexible garment assembly configured to be worn around the user's waist. The system additionally includes an anchor coupled to the flexible garment assembly at a plurality of attachment points, the anchor having a higher rigidity than the flexible garment assembly. The system additionally includes a first strap secured to the anchor and configured to selectively couple the socket to the flexible garment assembly via the anchor wherein the anchor is configured to transfer to a distributed area of the flexible garment assembly a force applied to the anchor by the socket.

### BACKGROUND

This application is directed to a system for attaching a prosthetic limb to a user's lower torso. In particular, this application is directed to a system including a socket configured to receive a leg amputation site, and a flexible garment to be worn around the user's waist and coupled to the socket.

One of the challenges in attaching a prosthetic limb to an individual is devising attachment systems that provide a secure attachment while facilitating user comfort. What is needed is a system that achieves these goals.

### SUMMARY

According to an embodiment, a system for attaching a prosthetic limb to a user's lower torso includes a socket shaped to receive therein a leg amputation site of the user. The system also includes a flexible garment assembly configured to be worn around the user's waist. The system additionally includes an anchor coupled to the flexible garment assembly at a plurality of attachment points, the anchor having a higher rigidity than the flexible garment assembly. The system further includes a first strap secured to the anchor and configured to selectively couple the socket to the flexible garment assembly via the anchor wherein the anchor is configured to transfer to a distributed area of the garment assembly a force applied to the anchor by the socket.

According to another embodiment, a mounting system for supporting attachment of a prosthetic limb to a leg amputation site of a user includes a flexible garment assembly configured to be worn around the user's waist. The system also includes an anchor coupled to the flexible garment assembly at a plurality of attachment points, the anchor having a higher rigidity than the garment assembly. The system additionally includes a strap secured to the anchor and configured to selectively couple the flexible garment assembly to a socket portion of the prosthetic limb that is shaped to receive therein the leg amputation site.

According to another embodiment, a system for attaching a prosthetic limb to a user's lower torso includes a socket shaped to receive therein a leg amputation site of the user. The system also includes a flexible garment assembly comprising fabric configured to be worn around the user's waist and surround the leg amputation site of the user; and a resilient material coupled to the fabric at the leg amputation site. The system also includes an anchor coupled to the flexible garment assembly at the leg amputation site. The anchor has a greater rigidity than the flexible garment assembly. The system further includes a first strap secured to the anchor and configured to selectively couple the socket to the flexible garment assembly via the anchor; and a second strap secured to the socket, and configured to surround the user's waist at an opposite side of the leg amputation site. The flexible garment assembly of the system is configured to retract against the user's lower torso. Additionally, the anchor of the system is configured to distribute a force applied to the anchor by the socket through a distributed area of the flexible garment assembly.

These and other objects, features, and characteristics of the invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. In one embodiment of the invention, the structural components illustrated herein are drawn to scale. It is to be expressly understood, however, that the drawings are for the purpose of illustration and description only and are not a limitation of the invention. In addition, it should be appreciated that structural features shown or described in any one embodiment herein can be used in other embodiments as well. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DISCUSSION OF THE DRAWINGS

FIG. 1 illustrates a side view of a system, including a socket assembly and a flexible garment assembly, for attaching a prosthetic limb to a user's lower torso according to an embodiment.

FIG. 2 illustrates a frontal view of the socket assembly of FIG. 1.

FIG. 3 illustrates an opposing side view of the socket of FIG. 1.

FIG. 4 illustrates a rear view of the garment assembly of FIG. 1.

### DETAILED DESCRIPTION

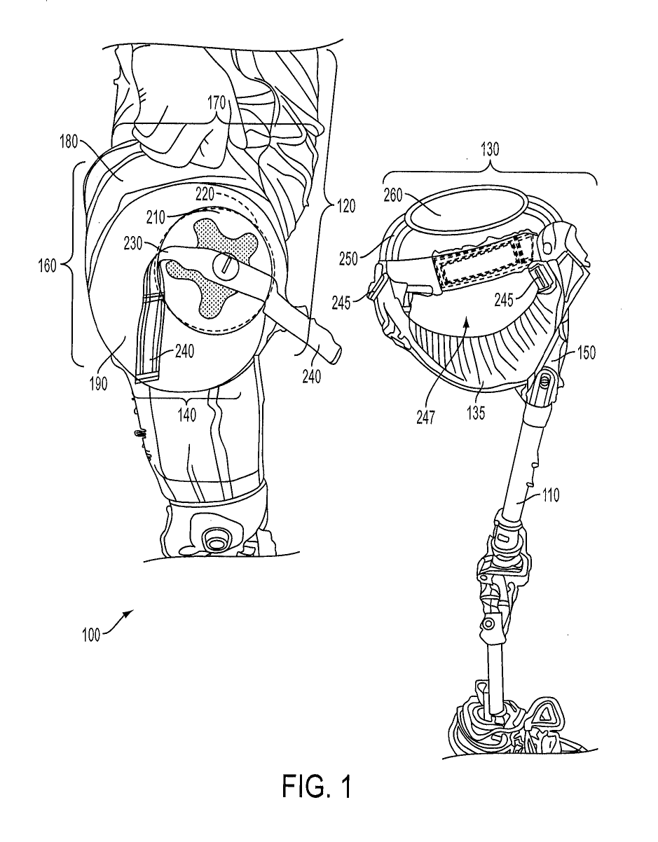

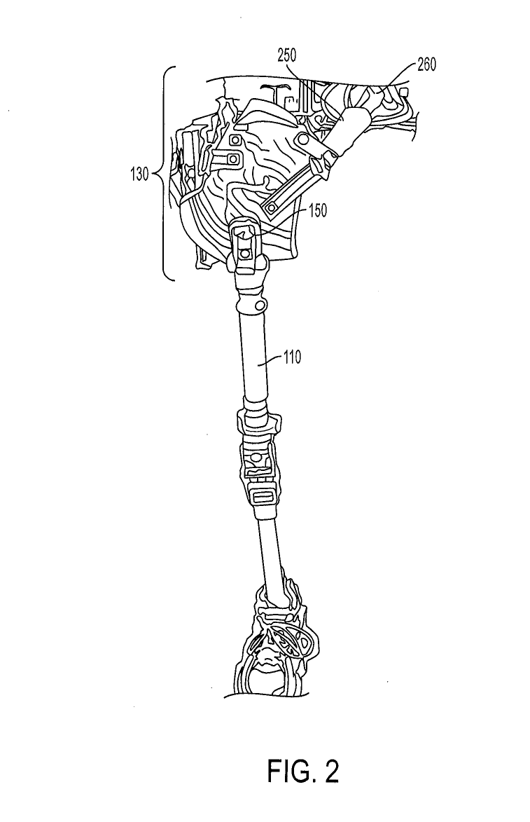

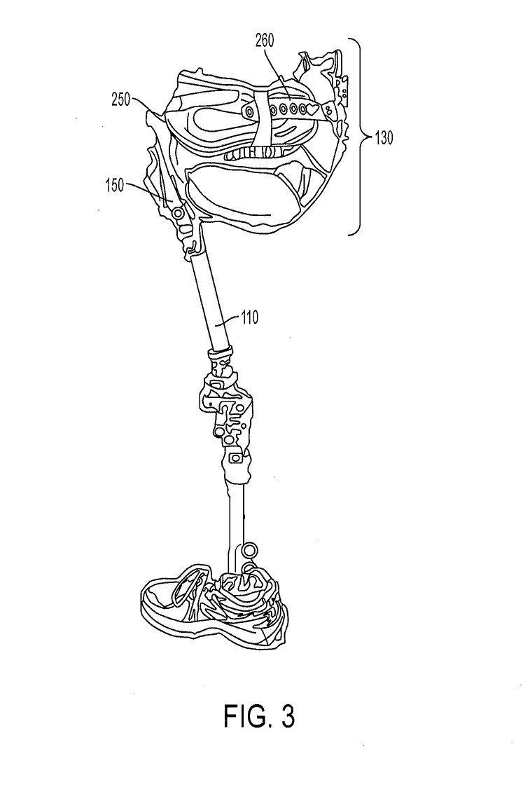

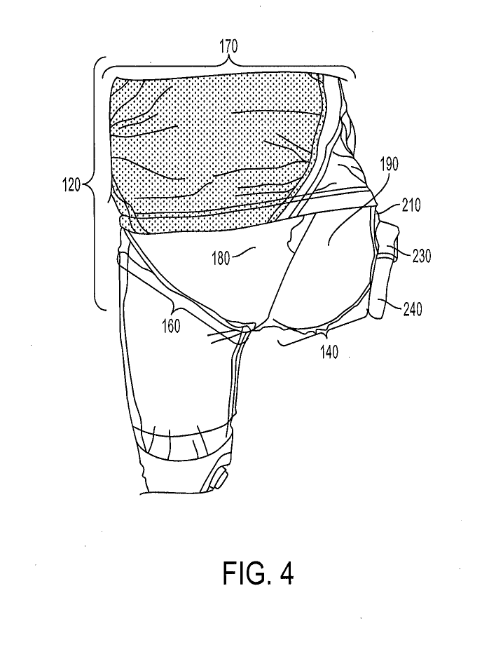

FIGS. 1-4 illustrate views of an embodiment of a system 100 for attaching a prosthetic limb 110 (i.e., a prosthetic leg) to a user's lower torso 120. The system 100 may include a socket assembly 130 configured to receive a leg amputation site 140 of the user. As discussed in greater detail below, the socket assembly 130 may include a socket 135 shaped to fit the leg amputation site 140. In various embodiments, the socket 135 may generally be formed of a durable and generally rigid material, which may support direct or indirect attachment of the prosthetic limb 110 thereto. For example, it may be appreciated that, in some embodiments, a prosthetic hip joint 150 may be coupled between the socket assembly 130 and the prosthetic limb 110, and may facilitate rotational movement of the prosthetic limb 110. In some embodiments, the socket assembly 130, including (for example) the socket 135 thereof, may comprise carbon fiber or other lightweight but durable material. In other embodiments, the socket assembly 130 and structures appurtenant thereto may comprise metal, plastic, silicone, or any combination thereof. In some embodiments, the socket 135 may itself comprise an assembly of components, which may be coupled together through any appropriate mechanism. For example, portions of the socket 135 may be coupled together, either through rigid or flexible connections, to generally surround the leg amputation site 140. In other embodiments, the socket 135 may be generally formed from a single molded material (e.g., a curved piece of carbon fiber or other generally rigid material) shaped to fit the leg amputation site 140.

The system 100 may also include a flexible garment assembly 160 configured to be worn around the user's waist 170, and facilitate the attachment of the socket assembly 130 (and the prosthetic limb 110 attached thereto) to the user. It may be appreciated that in some embodiments, the flexible garment assembly 160 may be custom tailored to surround the leg amputation site 140 of the user. In other embodiments, the flexible garment assembly 160 may be tailored in standardized sizes including small, medium, large, and any gradation thereof. In an embodiment, the flexible garment assembly 160 may comprise an elastic fabric 180, which may cause the flexible garment assembly 160 to be configured to retract against the user's lower torso 120. The elastic fabric 160 may be of any suitable construction or configuration, including but not limited to spandex, tricot, or nylon. Other fabrics or materials configured to snugly retract against the user's lower torso 120 may alternatively be utilized, including but not limited to latex or elastomers, which may be utilized alone or in combination with generally inelastic fabrics such as but not limited to cotton or polyester.

In some embodiments, a resilient material 190 may be coupled to the elastic fabric 180, which may increase the durability of the elastic fabric 180, and may allow for a greater distribution of forces across the elastic fabric 180. In an embodiment, the elasticity of the elastic fabric 180 may be formed from a generally inelastic fabric combined with the resilient material 190 therein. In an embodiment, the resilient material 190 may include silicone resin. Other resilient materials 190 may additionally or alternatively be utilized in various embodiments. For example, other natural or synthetic rubbers may be combined with the elastic fabric 180, or may be combined with a generally inelastic fabric to form the elastic fabric 180. It may be appreciated that in some embodiments, the resilient material 190 may be injected into the elastic fabric 180 around the leg amputation site 140. For example, in an embodiment the elastic fabric 180 may be stretched over a mold that may be shaped to match the contours of the leg amputation site 140 and/or the socket 135. The resilient material 190 may then be injected or otherwise applied to the elastic fabric 180, allowing the elastic fabric 180 at the leg amputation site 140 to generally hold the shape of the leg amputation site 140 (and/or the socket 135), while still retracting towards the user's lower torso 120. In some embodiments, by shaping the resilient material 190 to match the contours of the socket 135, forces from the socket assembly 130 may be more evenly transferred to the user's lower torso 120 via the resilient material 190.

In an embodiment, the flexible garment assembly 160 may additionally include an anchor 210 configured to further distribute forces associated with movement of the prosthetic limb 110 across the flexible garment assembly 160. In an embodiment, the anchor 210 may be secured to the resilient material 190 and/or the elastic fabric 180 by one or more attachment points 220. It may be appreciated that in some embodiments, the attachment points 220 may be at the perimeter of the anchor 210. For example, the attachment points 220 may comprise stitching the anchor 210 to the elastic fabric 180 and/or the resilient material 190 along the perimeter of the anchor 210. In other embodiments, the attachment points 220 may be distributed over a mating area between the anchor 210 and an associated region of the flexible garment assembly 160. For example, the attachment points 220 may comprise stitching positioned intermittently across the surface of the anchor 210. In another embodiment, the attachment points 220 may comprise adhesive bonding the anchor to the elastic fabric 180 and/or the resilient material 190, either at an edge or perimeter thereof, intermittently across an intermediate surface of the anchor 210, or across the entire intermediate surface of the anchor 210. In an embodiment, the anchor may be positioned at least partially between the elastic fabric 180 and the resilient material 190, such that the resilient material 190 assists in securing the anchor 210 into the flexible garment assembly 160.

It may be appreciated that the mating area and the anchor 210 may vary in size across embodiments. Additionally, the shape of the anchor 210 may also vary across embodiments. In various non-limiting examples, the mating area could cover at least 3, 4, 5, 6, 7, 8, 9, 10, 15, 20, 25, 30, and/or 40 square inches defined within a polygon connecting the plurality of attachment points 220. The anchor 210 may generally have the shape of a circle, such that the polygon is approximately circular. The anchor 210 may have any other polygonal or irregular shape.

To distribute the forces associated with moving the prosthetic limb 110 across the flexible garment assembly 160, the anchor 210 may have a higher rigidify than the elastic fabric 180 and/or the resilient material 190 of the flexible garment assembly 160. It may be appreciated that in some embodiments, the anchor 210 may have a rigidity that far surpasses the rigidity of the flexible garment assembly 160. For example, in some embodiments the anchor 210 may be generally resistant to deformation (e.g., may be stiff). In other embodiments, the anchor 210 may have a rigidity that is slightly higher than the rigidity of the flexible garment assembly 160, but may deform with some difficulty so as to somewhat track the deformation of the flexible garment assembly 160. The anchor 210 may be formed of any appropriate material, which may depend on the rigidity or other material characteristic desired to distribute forces across its structure. In some embodiments, the anchor 210 may comprise carbon fiber. In other embodiments, the anchor 210 may additionally or alternatively comprise plastic, silicone, and/or any other materials that may have a generally higher rigidity than the material of the flexible garment assembly 160.

To assist in securing the socket assembly 130 to the anchor 210 (and thus, the flexible garment assembly 160), the system 100 may further include a first strap 230 secured to the anchor 210. As shown, in an embodiment, the first strap 230 may be fixed to the anchor 210 by a fastener 235 in a manner that facilitates utilizing the first strap 230 to selectively couple the socket assembly 130 to the anchor 210. Specifically, movement of the leg amputation site 140 may cause movement of the anchor 210 coupled thereto (as the anchor 210 is fixed relative to the leg amputation site 140 by the remainder of the flexible garment assembly 160). The movement of the anchor 210 may cause movement of the socket assembly 130, secured to the anchor 210 by the first strap 230, as described below. Such movement of the socket assembly 130 may apply a force to the first strap 230, which may be transferred to the anchor 210 via the fastener 235. As noted above, the anchor 210 may then distribute the force from the first strap 230 and the fastener 235 across a distributed area of the flexible garment assembly 160.

The first strap 230 may be of sufficient length so as to form a loop engageable with the socket assembly 130. In some embodiments, the loop may be of an adjustable size. Adjustment of the loop may be associated with the configuration of the strap. For example, it may be appreciated that in some embodiments, the first strap 230 may form a loop of adjustable size by containing ratchet portions on opposing ends of the first strap 230. In other embodiments, such as that illustrated, the first strap 230 may form a loop of adjustable size by containing hook and loop connection portions 240. In various embodiments, the socket assembly 130 may contain anchor portions configured to engage the first strap 230, for coupling the first strap 230 to the socket assembly 130.

As shown in the illustrated embodiment, the socket assembly 130 includes a pair of strap anchors 245, which are spaced from one another, with an opening 247 formed in the socket 135 there between. Opposing ends of the first strap 230 may be received through apertures in the strap anchors 245, and then secured to each other by the hook and loop connectors 240 associated with each end. In an embodiment, the engagement between the first strap 230 and the socket assembly 130 may be tightened or loosened by adjusting placement of the hook and loop connectors 240 to one another. Where the first strap 230 is configured for other engagements mechanisms (e.g., ratcheting action), other mechanisms for modifying the engagement may alternatively be utilized. It may also be appreciated that while in the illustrated embodiment a single anchor 210 and a single first strap 230 are provided, in other embodiments additional anchors 210 and associated first straps 230 may be utilized to secure the socket assembly 130 to the flexible garment assembly. It may likewise be appreciated that while in some such embodiments the plurality of first straps 230 may be coupled to the same pair of strap anchors 245, or may be coupled to additional strap anchors 245 on the socket assembly.

By having the opening 247 formed in the socket 135, in particular placed to generally expose a portion of the anchor 210 when the leg amputation site 140 is received in the socket 135, breathability of the adjacent skin and other user comfort around the leg amputation site 140 may be achieved. For example, where the elastic fabric 180 is generally designed to wick sweat away from the user's body, evaporation of the sweat may be achieved with greater success when the elastic fabric is exposed at the opening 247, rather than being constrained by the denser material of the socket 135. Additionally, it may be appreciated that by reducing the structure of the socket 135 surrounding the leg amputation site 140, the socket assembly 130 may be a lighter weight, further facilitating lifting and movement of the prosthetic limb 110 by the user.

In some embodiments, to further secure the socket assembly 130 to the user, a second strap 250 may extend from the socket assembly 130 and be configured to loop around the user's waist, holding the socket assembly 130 to the user from a side of the user distal to the leg amputation site 140. As such, with the first strap 230 secured to the strap anchors 245 on one side of the user's lower torso 120, and the second strap 250 holding against the user's lower torso 120 from an opposite side of the user's lower torso 120, the socket assembly 130 may be supported from both sides of the user. Adjustment of the second strap 250 may be by any appropriate mechanism. For example, in some embodiments, the second strap 250 may have a length adjustable with a ratchet 260, such as is in the illustrated embodiments. In other embodiments, the second strap 250 may have a length adjustable with hook and loop connections. Adjustment of the second strap 250 may facilitate desired tightening of the socket assembly 130 from the side of the user opposite of the leg amputation site 140.

As shown in the illustrated embodiment, in some embodiments the socket assembly 130 may include a support structure 255 coupled to the second strap 250. It may be appreciated that the support structure 255 may be configured to snugly engage the waist 170 of the user. For example, the support structure 255 may include padding or other user-engageable materials, and may be configured to be positioned above the user's remaining hip. In an embodiment, adjustment of the second strap 250 may facilitate desired placement of a support structure 255. For example, by loosening the second strap 250 (i.e., expanding the size of the loop formed by the second strap 250 around the user's waist 270) the support structure 255 may be positioned higher on the user's waist 170. In some embodiments, the support structure 255 may comprise a curved portion configured to snugly support the user's waist 170. In some embodiments, the curved portion may be curved in both a horizontal and a vertical orientation (e.g., having a saddle-shape), so as to match contours of the user's waist 170 above the hip. While in some embodiments the support structure 255 may be selected from one of a variety of standard sizes, in other embodiments the support structure 255 may be custom sized for the user.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A system for attaching a prosthetic limb to a user's lower torso comprising:
a socket shaped to receive therein a leg amputation site of the user;
a flexible garment assembly configured to be worn around the user's waist;
an anchor coupled to the flexible garment assembly, the anchor having a greater rigidity than the flexible garment assembly; and
a first strap secured to the anchor and configured to selectively couple the socket to the flexible garment assembly via the anchor;
wherein the anchor is configured to transfer to a distributed area of the flexible garment assembly a force applied to the anchor by the socket.

2. The system of claim 1, further comprising a prosthetic limb portion coupled to the socket.

3. The system of claim 1, wherein the first strap forms a loop of adjustable size with a ratchet.

4. The system of claim 1, wherein the first strap forms a loop of adjustable size with hook and loop connections.

5. The system of claim 1, further comprising a second strap secured to the socket and configured to secure the socket to the user's waist from a side of the user distal to the leg amputation site.

6. The system of claim 5 wherein the second strap has a length adjustable with a ratchet.

7. The system of claim 5 wherein the second strap has a length adjustable with hook and loop connections.

8. The system of claim 1 wherein the socket comprises carbon fiber.

9. The system of claim 1 wherein the socket couples to a prosthetic hip joint.

10. The system of claim 1, wherein the flexible garment assembly is custom tailored to surround the leg amputation site of the user,

11. The system of claim 1, wherein the flexible garment assembly comprises an elastic fabric.

12. The system of claim 11, wherein the elastic fabric is configured to retract against the user's lower torso.

13. The system of claim 11, further comprising silicone resin coupled to the elastic fabric.

14. The system of claim 13, wherein the silicone resin is injected into the elastic fabric at the leg amputation site.

15. The system of claim 14, wherein the silicone resin is shaped to match contours of the socket.

16. A mounting system for supporting attachment of a prosthetic limb to a leg amputation site of a user, the system comprising:
a flexible garment assembly configured to be worn around the user's waist;
an anchor coupled to the flexible garment assembly at a plurality of attachment points, the anchor having a higher rigidity than the flexible garment assembly, and
a strap secured to the anchor and configured to selectively couple the flexible garment assembly to a socket portion of the prosthetic limb that is shaped to receive therein the leg amputation site.

17. The system of claim 16, wherein the flexible garment assembly is custom tailored to surround the leg amputation site of the user.

18. The system of claim 16, wherein the flexible garment assembly comprises an elastic fabric configured to retract against the user's torso.

19. The system of claim 18, further comprising silicone resin coupled to the elastic fabric.

20. A system for attaching a prosthetic limb to a user's lower torso comprising:
a socket shaped to receive therein a leg amputation site of the user;
a flexible garment assembly comprising:
fabric configured to be worn around the user's waist and surround the leg amputation site of the user; and
a resilient material coupled to the fabric at the leg amputation site;
an anchor coupled to the flexible garment assembly at the leg amputation site, the anchor having a greater rigidity than the flexible garment assembly; and
a first strap secured to the anchor and configured to selectively couple the socket to the flexible garment assembly via the anchor; and
a second strap secured to the socket, and configured to surround the user's waist at an opposite side of the leg amputation site;
wherein the flexible garment assembly is configured to retract against the user's lower torso; and
wherein the anchor is configured to distribute a force applied to the anchor by the socket through a distributed area of the flexible garment assembly.
